(19) United States
(12) Patent Application Publication
(10) Pub. No.: US 2022/0355047 A1
(43) Pub. Date: Nov. 10, 2022

(54) Insufflation Tube for Laparoscopy with Heating Element, Humidifying Agent and Device for Determining the Moisture Content

(71) Applicant: W.O.M. World of Medicine GmbH, Berlin (DE)

(72) Inventor: Bernd Müller, Berlin (DE)

(73) Assignee: W.O.M. World of Medicine GmbH, Berlin (DE)

(21) Appl. No.: 17/737,512

(22) Filed: May 5, 2022

Related U.S. Application Data
(60) Provisional application No. 63/184,895, filed on May 6, 2021.

(51) Int. Cl. A61M 13/00

(52) U.S. Cl. CPC A61M 13/003; A61M 2205/3653; A61M 2205/3327; A61M 2205/3368; A61M 2205/18

(57) ABSTRACT

The present disclosure relates to an insufflator with insufflation tube with integrated heating element and humidifier for laparoscopy, whereby measurement of the moisture content is possible.

### CROSS REFERENCE TO RELATED APPLICATIONS

This application claims priority to U.S. Provisional Patent Application No. 63/184,895 filed on May 6, 2021 and entitled Insufflation Tube for Laparoscopy with Heating Element, Humidifying Agent and Device for Determining the Moisture Content, the entire contents which hereby incorporated by reference.

### FIELD OF THE DISCLOSURE

The present invention relates to an insufflator with insufflation tube with integrated heating element and humidifier for laparoscopy, whereby measurement of the moisture content is possible.

### BACKGROUND AND STATE OF THE ART

Laparoscopy is a medical procedure that allows visual inspection of the abdominal cavity and the organs within it. For this purpose, small skin incisions (0.3 to 2 centimeters) are usually made in the abdominal wall and a trocar is inserted through them, which in turn can accommodate an optical device. With the help of a special endoscope (laparoscope), the abdominal cavity can be viewed. In diagnostic laparoscopy, the abdominal cavity is inspected only visually; in therapeutic laparoscopy, surgical procedures can also be performed.

Usually, at the beginning of laparoscopy, the abdominal cavity is first filled with gas to create a pneumoperitoneum. Various gases have been used for this purpose, such as air, nitrogen, or carbon dioxide (CO2). The use of carbon dioxide gas has been particularly successful. It has been found that, especially during longer laparoscopic procedures, it is useful to heat the introduced gas on the one hand and to humidify it on the other. Gas warming serves to prevent the patient from cooling down, as well as to avoid a diffuse feeling of pain on the part of the patient, which is probably a consequence of local cooling due to the entry of cold gas.

Humidification serves to prevent drying of the internal abdominal surfaces in order to avoid the resulting cooling.

Suggestions for this have already been made in the state of the art. For example, the German patent specification DE 19510710 describes a device which provides a means for adjusting the gas humidity (for example a sponge) and which can optionally contain an additional heating element.

DE 10 2013 000492 A1 describes a tube with an integrated heating element for laparoscopy, which also contains a humidifying agent. According to this publication, the humidifying agent is moistened with water before an operation. Depending on the water absorption of the material described there, the gas volume flow and the duration of the operation, it may be necessary to rewet the humidifying agent intraoperatively. Since the evaporation rate of the water, depends on a number of parameters, so far it can only be estimated when a refill is possible. Alternatively, designs are described which arrange a humidity sensor for determining the gas humidity in the gas channel. However, this has several disadvantages. First, the humidity sensor must be electrically connected, which makes the design of the filter interface more complicated. Furthermore, the moisture sensor generates a not inconsiderable flow resistance in the gas channel. This leads to a lower flow rate, which contradicts the current flow requirements.

Another device for humidifying gases in medical technology is described in DE 3617031A1 (priorities: NZ 21263, NZ 215123 and NZ 214694). Within the scope of a hose system that is complex to manufacture, a hose that is always filled with water is provided. Water vapor is released to the gas via a microporous hose wall. A sensor monitors the water temperature.

A further solution known from the prior art is to use a humidity sensor. One such solution can be a sensor that measures the temperature profile during heating, as described in WO 2017/157 365 A1.

Another way to determine residual moisture without a moisture sensor is described in U.S. Pat. No. 8,836,521 “Hydration alert”: The heating work performed (electrical work) is determined and used to trigger an alert to the user to refill the humidification medium. There is also described to use the total amount of insufflated expansion medium to trigger a refill alarm. Disadvantage of the described solutions is a high expenditure for sensors and devices in the device, linked with an only indirect determination of the humidification medium present in the humidification mesh.

The present invention is intended to overcome the disadvantages of the solutions known from the prior art.

### SUMMARY OF THE DISCLOSURE

The present disclosure is directed to an insufflation device for use in medical technology that includes, inter alia, an insufflator for gas supply and an insufflation tube. The insufflation tube contains a humidifying material in its interior, the humidifying material being in contact with a heating element. The heating element preferably consists of a wire which can be activated by applying a current. It is envisioned that the insufflation tube includes two wires insulated from each other, which together form a capacitor, the impedance of which depends on the humidity of the humidifying material.

Preferably, at least one of the wires is electrically insulated from the other and is arranged on: the outer wall of the insufflation tube, inside the wall of the insufflation tube, on the inner wall of the insufflation tube or inside the insufflation tube.

It is envisioned that the insufflation tube may further include a temperature sensor. In an embodiment, the temperature sensor is arranged at the patient-side end of the insufflation tube.

In certain constructions, at least one of the capacitor forming wires, is formed by a connecting a cable of the temperature sensor.

In a preferred embodiment, at least one of the capacitor-forming wires, is formed by a heating wire.

The present disclosure is also directed to a method for measuring the water content of a humidifying material, which is located in an insufflation tube of an insufflator, through which a gas flows. In an embodiment of the disclosure method, a high-frequency voltage is applied to the two wires forming a capacitor; the impedance of the capacitor is determined and the humidity of the humidifying material is determined from the impedance.

In an embodiment of the disclosure method, a first impedance is measured when the humidifying material is dry; a second impedance is measured when the humidifying material contains the maximum amount of water and further frequent impedance measurements are taken during insufflation.

In an embodiment of the disclosure method, an alarm signal is triggered when humidity of the humidifying material falls below a preset threshold value. In certain embodiments, the preset threshold value corresponds to 50%, 40%, 30%, 20%, 10% or 5% of the maximum humidity.

### BRIEF DESCRIPTION OF THE FIGURES

Exemplary embodiments of the present disclosure are further described with reference to the appended figures. It is to be noted that the various features, steps, and combinations of features/steps described below and illustrated in the figures can be arranged and organized differently to result in embodiments which are still within the scope of the present disclosure.

To assist those of ordinary skill in the art in making and using the disclosed assemblies, systems and methods, reference is made to the appended figures, wherein:

FIG. 1 schematically shows the structure of an insufflation tube according to an embodiment of invention wherein an empty tube is shown at the top.

FIG. 2 shows an embodiment of the invention in which the capacitor-forming wires are formed by the leads of a temperature sensor.

FIG. 3 shows an alternative embodiment of the invention in which one of the capacitor-forming wires is formed by a heating wire. In an optional variant, the temperature can also be measured by the heating wire (analogous to WO 2014/111083 A1).

FIG. 4 shows an example of the measurement cycle and the results: The hose used as an example is first measured in dry form and shows an impedance of 100 ohms. At time t=0 s, it is moistened with 10 ml of water (distilled), and the impedance drops to 10 ohms. By passing gas (here: CO2) with simultaneous heating to 39° C., the water evaporates in the course of time, respectively in the course of the gas flow (here: constant gas flow of 10 l/min). After 2001 have passed through, the water is practically completely used up, the humidification material is dry again, so that the original impedance of 100 Ohm is measured again (approximately). It can be seen that the measured absolute values differ when an isotonic saline solution is used instead of (distilled) water (dashed line). However, the course of the measurement curve is similar, so that a moisture measurement is also possible in this case.

### DETAILED DESCRIPTION OF PREFERRED EMBODIMENTS

The disclosure teaches an embodiment of a heated hose with which the permittivity of the humidifying material can be measured. The basic principle of the solution according to the invention is a construction of the heating hose analogous to a capacitor.

Capacitors are known from electrical engineering as passive components. In principle, capacitors consist of two electrically conductive surfaces (electrodes) that are separated from each other by an insulating material, the dielectric. Common designs of such capacitors contain plate-shaped electrodes (capacitor plates). The electro-technical properties of the capacitors are determined not only by the area, volume and spacing of the electrodes from one another, but also in particular by the permittivity (ε) of the dielectric located between the plates.

It is also known that a capacitor forms a resistance when an alternating current (AC) is applied. This AC resistance is also called impedance and can be described as complex AC resistance. The impedance can be measured by methods described in the prior art, in particular by measuring the resistance of an applied alternating current. It is relevant to the present invention that the impedance of a capacitor changes by changing the permittivity (ε) of the dielectric.

In the present case, the dielectric consists primarily of the humidifying material and its water content (“humidity”). Due to the electrical conductivity of water, the electrodes must be electrically insulated from each other. For this purpose, at least one of the electrodes is sheathed with an electrical insulation.

For the purpose of application according to the invention, namely the measurement of humidity within an insufflation tube, which in turn contains a heating element and a humidified humidification material, in the simplest case two wires can be arranged as electrodes forming the capacitor described above. The wires may be arranged inside the hose lumen, and the wires may in particular be attached (e.g., glued or welded) to the inner wall of the hose. Fixation is recommended because otherwise the impedance may change with the possible movement of the heated hose. In further possibilities, the wires may be incorporated (e.g. cast) into the hose wall or attached (e.g. glued) to the hose outer wall. An arrangement within the hose is preferred. The wires may be arranged in a largely straight line (parallel to the orientation of the hose). Alternatively, they may be arranged helically along the hose wall. It is crucial for the function according to the invention that the moistening material is located between the wires. The two wires, at least one of which must be insulated, thus form a capacitor whose impedance (at constant frequency) depends on the properties of the dielectric. When the degree of humidity of the dielectric changes, there are changes in impedance that can be measured.

Referring now to the drawings, wherein like parts are marked throughout the specification and drawings with the same or similar reference numerals. Drawing figures are not necessarily to scale and in certain views, parts may have been exaggerated for purposes of clarity.

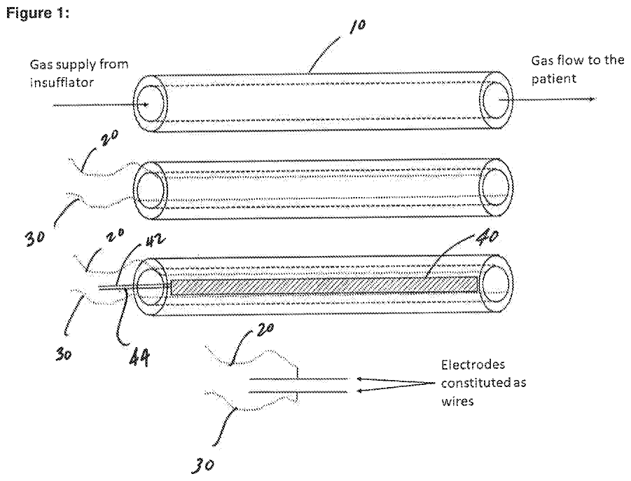

FIG. 1 schematically shows the structure of an insufflation tube according to an embodiment of invention. An empty tube 10 is shown at the top. Two wires, 20 and 30 respectively, are inserted into the tube and fixed to the tube wall (center). The connections to the insufflator are shown. In the bottom illustration, you can see the humidifier 40, which is coiled with the heating wire. The electrical heating wire connections 41/42 can also be seen.

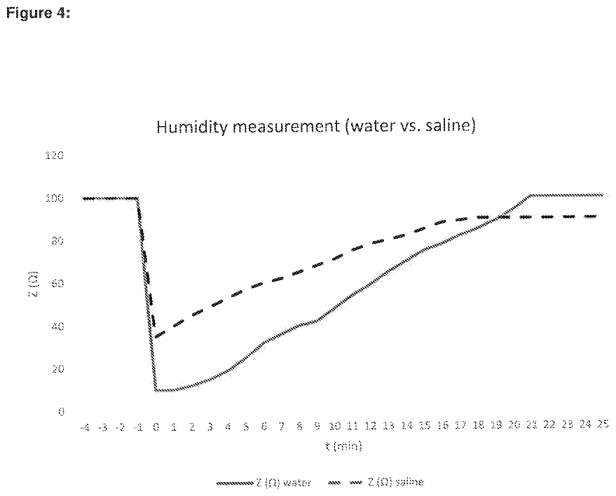

Investigations have shown that simple metal (e.g. copper) wires with a diameter of 0.1-1 mm are already sufficient to measure the change in dielectric properties of the humidifier (see below). FIG. 4 shows the measured change in impedance over the insufflation time.

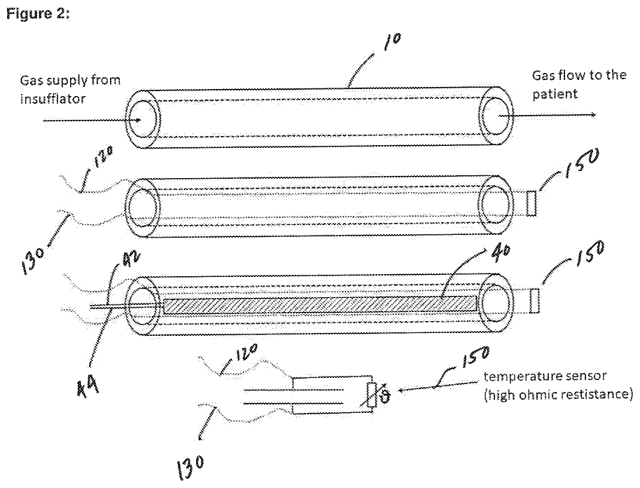

In another embodiment of the invention shown in FIG. 2, the capacitor-forming wires 120/130 are formed by the leads of a temperature sensor 150. In this embodiment, a common temperature sensor (e.g., DS18S20) is located in the tubing. In any case, it must be a high-impedance temperature-dependent resistor at the AC frequency applied for measurement, so that the capacitor is not short-circuited. A basic circuit diagram is shown in FIG. 2.

In this embodiment of the invention, temperature and moisture content cannot be measured simultaneously; the measurement of these properties takes place one after the other, in particular in continuous alternation. By applying a direct current to the two connecting cables, the resistance of the temperature sensor 150 can be measured, from which the temperature is then determined. By applying a high-frequency alternating current, the impedance of the capacitor formed can be determined, which is related to the humidity of the humidifying material.

The advantage of this embodiment is that the number of wires in the tube is limited. Also in this embodiment, the connecting wires for the temperature sensor can be arranged outside or inside the hose or in the hose wall.

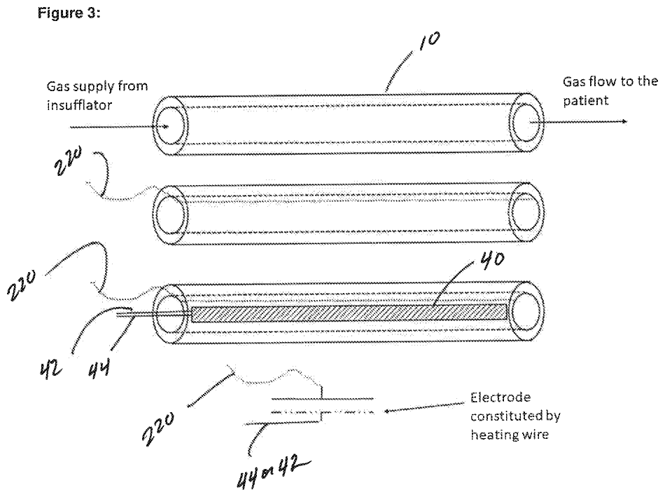

In a further alternative embodiment of the invention shown in FIG. 3, one of the wires forming the capacitor can also be formed by a heating wire 42 or 44. Insufflation hoses with heating wires 42/44 have been described, for example, in WO 2014/111083 A1. This embodiment also only allows heating or measurement alternating in time: By applying a direct current to the heating wire 42/44, the latter develops heat through the ohmic resistance, which heats the gas and the humidifying agent passing through. By applying a high-frequency alternating voltage to the heating wire on the one hand and a parallel wire on the other, the impedance of the capacitor formed by this can be determined and from this the humidity of the humidifying material can be determined.

In a preferred embodiment of the invention, the gas inflow into the insufflation tube takes place through a humidifying material formed as a braided tube, which in turn is positioned inside the insufflation tube. As described in WO 2014/111083 A1, the braided hose may also be sheathed with the humidification material and the heating wire. In either case, the gas flowing through the braided hose exits through the pores of the braided hoses' surface and is both heated and humidified. In this embodiment of the invention, it is essential that the braided tubing be made of a material that is non-conductive at the AC frequency applied for measurement.

If the heating wire is a material in which the resistance increases with temperature (e.g. a material with a positive or negative temperature coefficient (PTC or NTC), as described in WO 2014/111084 A1), then the temperature can also be measured via measuring the resistance of the heating wire. The details of such a temperature measurement are described in WO 2014/111084 A1, so that reference can be made to this. In such an embodiment of the invention, heating periods, measurement period for moisture measurement of the humidifying material and temperature measurement alternate periodically. The use of digital measuring sensors in such a setup is less recommendable, since the supply lines for digital temperature sensors as a bus system are susceptible to interference due to the irradiation of RF signals.

The measurement of the humidity of the humidifying agent according to the invention is carried out by measuring the impedance by means of an applied alternating current of the highest possible frequency (e.g. 100 kHz to 100 MHz). Since the impedance depends on the exact construction of the device according to the invention (among other things, distance of the capacitor-forming wires from each other, type of humidifying material, composition of the humidifying agent, geometrical arrangement of the wires, etc.), an impedance measurement must be carried out for each construction of such a tube. For this purpose, the hose construction shown below is first realized without humidifying agent (water) and the impedance of the capacitor formed is measured using conventional methods. Then, the humidifying material is humidified to the maximum by adding the humidifying agent (water). Using a dummy, the tube is then heated and gas flows through it. Meanwhile, the impedance is measured continuously or periodically and the course of the impedance is recorded. Due to the possible different designs of such insufflation hoses according to the invention, the absolute value of the impedance is comparatively unimportant. What is decisive is the course of the impedance in correlation with the degree of humidification of the humidification material.

FIG. 4 shows an example of the measurement cycle and the results: The tube used as an example is first measured in dry form and shows an impedance of 100 ohms. By moistening with 10 ml of water, the impedance drops to 10 ohms. By passing through gas (here: CO₂) with simultaneous heating to 39° C., the water evaporates in the course of time, or in the course of the gas flow (here: constant gas flow of 10 l/min). After 2001 have passed through, the water is practically completely used up, the humidification material is dry again, so that the original impedance of 100 Ohm is measured again (approximately). It can be seen that the measured absolute values differ when an isotonic saline solution is used instead of (distilled) water. However, the course of the measurement curve is similar, so that a moisture measurement is also possible in this case.

The main task of the present invention is to determine the condition of the humidifying agent with respect to its water content, i.e. the water content of the humidifying agent, without implementing the disadvantages mentioned at the beginning. The primary objective is to generate a refill alarm/signal, i.e., a signal that informs the user when a refill of water is necessary.

For example, an alarm signal can be triggered when the water content of the humidification material falls below a preset threshold. For example, the alarm signal may be triggered when a preset threshold corresponds to 50%, 40%, 30%, 20%, 10% or 5% of the maximum moisture content.

In the context of the present invention, the terms “water content of the humidifying agent” and “humidity of the humidifying agent” are considered synonymous.

1. An insufflation device for use in medical technology comprising
an insufflator for gas supply and an insufflation tube,
wherein the insufflation tube contains a humidifying material in its interior, the humidifying material being in contact with a heating element,
wherein the heating element consists of a wire which can be activated by applying a current,
wherein the insufflation tube comprises two wires insulated from each other, which together form a capacitor, the impedance of which depends on the humidity of the humidifying material.

2. The device according to claim 1, wherein at least one of the wires electrically insulated from each other is arranged on the outer wall of the insufflation tube, inside the wall of the insufflation tube, on the inner wall of the insufflation tube or inside the insufflation tube.

3. The device according to claim 1, wherein the insufflation tube further comprises a temperature sensor.

4. The device according to claim 2, wherein the temperature sensor is arranged at the patient-side end of the insufflation tube.

5. The device according to claim 3, wherein at least one of the capacitor forming wires, is formed by a connecting cable of the temperature sensor.

6. The device according to claim 1, wherein at least one of the capacitor-forming wires, is formed by a heating wire.

7. A method for measuring the water content of a humidifying material, which is located in an insufflation tube of an insufflator, through which a gas flows, wherein
a) a high-frequency voltage is applied to the two wires forming a capacitor
b) the impedance of the capacitor is determined
c) the humidity of the humidifying material is determined from the impedance.

8. The method according to claim 7 comprising
a first impedance measurement when the humidifying material is dry
a second impedance measurement when the humidifying material contains the maximum amount of water
further frequent impedance measurements during insufflation.

9. The method according to claim 7, characterized in that an alarm signal is triggered when humidity of the humidifying material falls below a preset threshold value.

10. The method according to claim 9, characterized in that the preset threshold value corresponds to 50%, 40%, 30%, 20%, 10% or 5% of the maximum humidity.
